(19) United States
(12) Patent Application Publication
(10) Pub. No.: US 2005/0095286 A1
(43) Pub. Date: May 5, 2005

(54) Extended release compositions

(75) Inventors: Stephen J. Kramer, Houston, TX (US); Louis F. Fabre, Houston, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Fabre Kramer Pharmaceuticals, Inc., Houston, TX (US)

(21) Appl. No.: 10/875,384

(22) Filed: Jun. 25, 2004

Related U.S. Application Data
(60) Provisional application No. 60/482,012, filed on Jun. 25, 2003.

(51) Int. Cl. A61K 31/506; A61K 9/20

(52) U.S. Cl. 424/464; 514/252.19

(57) ABSTRACT

A pharmaceutical composition is provided, which includes: at least one active ingredient selected from the group including adatanserin, a compound having formula I, a compound having formula II, abaperidone, pharmaceutically acceptable salt thereof, prodrug thereof, and a mixture thereof; at least one high or medium viscosity hydroxypropylmethylcellulose (HPMC); at least one high or medium viscosity hydroxyethylcellulose (HEC); wherein the HPMC and HEC are present in a HPMC/HEC weight ratio ranging from 1/0.5 to 1/1.5; wherein adatanserin, abaperidone, and the compounds having formulas (I) and (II) are defined herein.

### BACKGROUND OF THE INVENTION

1. Field of the Invention

The present invention is related to a pharmaceutical compositions having a sustained or extended release behavior.

2. Discussion of the Background

Hydrophilic gel forming matrix compositions are well known dosage forms to control the dissolution behavior of active ingredients. The mechanism by which the active ingredient is released starts with the hydration of the dosage form surface to form a gel structure. Simultaneously, the active ingredient at the composition surface dissolves in the dissolution medium. In the stationary phase, the dissolution medium continuously penetrates the gel structure, and the gel expands. The active ingredient dissolves in the dissolution medium and is transported to the outer layer of the gel. Meanwhile, erosion of the outer layers of the gel occurs. The release levels off, which is caused by the decreased concentration gradient of the active ingredient in the composition and the penetrated dissolution medium. This mechanism is described, for example, in Manford Robinson, The Theory and Practice of Industrial Pharmacy, 2nd edition, Chapter 14: “Sustained Action Dosage Forms”.

Hydrophilic polymers such as described above are mostly polysaccharide carriers. Examples of these include hydroxypropylmethylcellulose (HPMC), hydroxypropylcellulose (HPC), hydroxyethylcellulose (HEC), sodium carboxymethylcellulose (NaCMC) or combinations of these cellulose derivatives. Compositions of these type are described, e.g., in U.S. Pat. No. 4,871,548 and EP-A-0923934.

U.S. Pat. No. 4,871,548 discloses a controlled release dosage form that includes an active compound and a mixture of at least a low viscosity cellulose ether and a high viscosity cellulose ether. EP-A-0923934 discloses a modified release matrix composition of cefaclor and cephalexin that includes 5-35% of a mixture of hydrophilic polymers of different grades, wherein the hydrophilic polymers include about 0.1-20% by weight of medium viscosity hydroxypropyl methylcellulose and about 0.1-20% of low viscosity hydroxypropylcellulose.

Although the compositions mentioned above are described as sustained release compositions in general, this sustained release only appears when the concentration of salts, the ion-strength, in the dissolution medium is low. The release rate of the active ingredient from the above mentioned compositions may be substantially dependent on the ion-strength. A high ion-strength may lead to “dose-dumping”, wherein all or most of the active ingredient is released in a very short time. Dose-dumping can undesirably lead to dangerously high blood levels of the active ingredient. Since high ion-strengths often occur after eating and since patients typically take their medications just after meals there is a risk that ion-strength dependent compositions undesirably result in a dose-dumping of the active ingredient instead of a more desirable sustained release.

WO 98/47491 describes a sustained release composition wherein the release of the active ingredient is controlled with a combination of two polymers having opposing wettability characteristics: one polymer is more hydrophobic and the other polymer is more hydrophilic. In this composition dose dumping can only be prevented by coating with an enteric coating, however.

The compound, abaperidone, shows an interesting profile as a neuroleptic, has a high therapeutic index and a high affinity to 5HT₁ₐ-receptors, which also makes it potentially useful in the treatment of anxiety. Biochemical assays have revealed that these compounds exhibit an enhanced action on the receptors involved in neuroleptic (D₂ and 5HT₂) and anxiolytic (5HT₁ₐ) actions (U.S. Pat. No. 5,736,558; B. A. McMillen et al., “Drug Dev. Res.”, 1988, 12, 53-62, both incorporated herein by reference).

The compound, adatanserin, has demonstrated selectivity at the 5-HT_{1A} and 5-HT₂ versus D₂ receptor binding sites and is useful as a potential anxiolytic-antidepressant agent. In addition, it has a high affinity for the 5-HT_{1A} and D₂-receptor binding sites and is useful as a mixed antipsychotic-anxiolytic agent. See, e.g., U.S. Pat. No. 5,010,078.

Compounds described in U.S. Pat. No. 5,077,293 and U.S. Pat. No. 5,300,506 inhibit the re-uptake of endogenous serotonin. Selective inhibitors of serotonin uptake are effective for the treatment of mental depression and have been reported to be useful for treating chronic pain (see: R. W. Fuller, “Pharmacologic Modification of Serotonergic Function: Drugs for the Study and Treatment of Psychiatric and Other Disorders”, J. Clin. Psychiatry 47:4 (Suppl.) Apr. 1986, pp. 4-8), incorporated herein by reference. These compounds are particularly suitable in the following disorders: obsessive-compulsive disorder, feeding disorder, anxiety disorder, panic disorder, social disorder and social phobia. In addition, they inhibit norepinephrine re-uptake, and blockade of endogenous norepinephrine re-uptake is also a mechanism through which it is believed that various antidepressant agents exert their therapeutic effect (see: “Antidepressants: Neurochemical, Behavioral, and Clinical Perspectives”, edited by S. J. Enna, J. B. Malick and E. Richardson, (1981), Raven Press, New York, pp. 1-12), the entire contents of which are hereby incorporated by reference.

Serotonin has been linked to the pathophysiology of migraine by accumulating evidence including increased excretion of serotonin metabolites following a migraine attack and a reduction in the serotonin content of blood platelets during the migraine headache. This latter effect appears to be specific for migraine and not a result of pain or stress. (Anthony, et al., “Plasma serotonin in migraine and stress”, Arch. Neurol. 1967, 16: 544-552, the entire contents of which are hereby incorporated by reference). More importantly, intramuscular injection of reserpine lowers plasma serotonin and induces a typical migraine-type headache in migraine sufferers. This induced headache can be alleviated by slow I.V. injection of serotonin creatinine sulfate. (Kimball, et al., “Effect of serotonin in migraine patients”, Neurology N.Y.), 1960, 10: 107-111, the entire contents of which are hereby incorporated by reference).

Although serotonin has been shown to be effective in treating migraine attacks, its use in migraine is precluded by its side-effects such as restlessness, nausea, faintness, hyperpnea, facial flushing and parasthesias. (Lance, et al., “The control of cranial arteries by humoral mechanisms and its relation to the migraine syndrome”, Headache, 1967, 7: 93-102), the entire contents of which are hereby incorporated by reference. For this reason more specific serotonin agents, which would treat the migraine without all of the other actions, are potentially useful antimigraine medicaments. Accumulating findings have led to the perception that compounds with selectivity for the 5-HT_{ID} sub-type of serotonin receptors would be clinically efficacious in the treatment of migraine.

### SUMMARY OF THE INVENTION

One object of the invention is to solve the problems associated with conventional compositions mentioned above.

Another object of the present invention is to provide a sustained release composition which is substantially independent from the ion-strength of the dissolution medium, which is typically the gastrointestinal fluid.

Another object of the present invention is to provide a sustained release composition which is substantially independent from the ion-strength of the dissolution medium, even when the composition is not coated.

Surprisingly and unexpectedly, these and other objects have now been achieved by the present invention, the first embodiment of which provides a pharmaceutical composition, which includes:

at least one active ingredient selected from the group including adatanserin, a compound having formula I, a compound having formula II, abaperidone, pharmaceutically acceptable salt thereof, prodrug thereof, and a mixture thereof,

at least one high or medium viscosity hydroxypropylmethylcellulose (HPMC);

at least one high or medium viscosity hydroxyethylcellulose (HEC);

wherein the HPMC and HEC are present in a HPMC/HEC weight ratio ranging from 1/0.5 to 1/1.5;

wherein the compound having the formula (I) is:

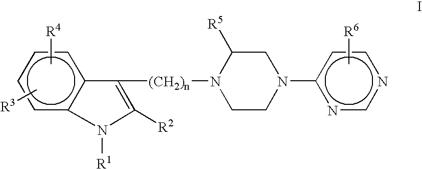

wherein R¹ is selected from the group including hydrogen, lower alkyl and aryl-lower alkyl, R² and R⁵ are each independently selected from the group including hydrogen and lower alkyl; R³ and R⁴ are each independently selected from the group including hydrogen, lower alkyl, lower alkoxy, lower alkylthio, carboxamide, halogen and trifluoromethyl; R⁶ is lower alkoxy, and n is the integer 2 or 3;

and wherein the compound of formula (II) is:

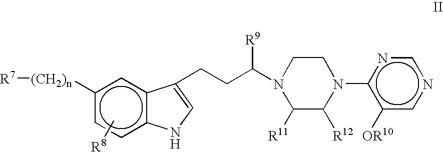

wherein R⁷ is selected from the group including hydrogen, halogen, lower alkyl, lower alkoxy, phenyl-substituted lower alkoxy, said phenyl being unsubstituted or bearing a lower alkyl substituent, amino, cyano, hydroxy, nitro, —OCH₂CN; —OCH₂CONR¹³R¹⁴; —SO₂NR¹³R¹⁴; —O₂CR¹⁵; —SO₂R¹⁵; —O₂CNR¹³R¹⁴; —COR¹⁴; —CO₂R¹⁵; —CONR¹³R¹⁴; —NR¹³CO₂R¹⁵; —NR¹³COR¹⁴; —NR¹³SO₂R¹⁵; and

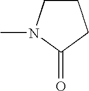

R⁸ is selected from the group consisting of hydrogen, halogen, lower alkyl, lower alkoxy and —CO₂R¹⁵;

R⁹, R¹¹, R¹² and R¹³ are each independently selected from the group including hydrogen and lower alkyl with the proviso that R⁹ is not hydrogen when the R⁷—(CH₂)ₙ moiety is hydrogen, halogen, lower alkyl, lower alkoxy or —CONH₂ and R⁸ is hydrogen, halogen or lower alkyl;

R¹⁰ is lower alkyl;

R¹⁴ is selected from the group including hydrogen, lower alkyl, R¹³-substituted phenyl-lower alkyl and trifluoromethyl;

R¹⁵ is selected from the group including lower alkyl and R¹³-substituted phenyl-lower alkyl; and

n is 0, 1, or 2.

Another embodiment of the present invention provides a method, which includes administering the above composition to a human.

Another embodiment of the present invention provides a method for treating or preventing at least one selected from the group including social phobia, general anxiety disorder, major depression, atypical depression, schizophrenia, psychotic disorder, and combinations thereof which includes administering the above composition to a subject.

Another embodiment of the present invention provides a method of preparing the above composition, which includes: (1) preparing a core by compressing a mixture containing one or more of the active ingredients and a mixture of the high or medium viscosity hydroxypropylmethylcellulose (HPMC) and the high or medium viscosity hydroxyethylcellulose (HEC); and (2) optionally coating the core.

### DESCRIPTION OF THE PREFERRED EMBODIMENTS

Various other objects, features and attendant advantages of the present invention will be more fully appreciated as the same becomes better understood from the following detailed description of the preferred embodiments of the invention.

The present invention provides a pharmaceutical composition with a substantially sustained or extended release behavior which is preferably independent of the ion-strength of the dissolution medium, e.g., the gastrointestinal fluid. Sustained or extended release is defined as a (gradual) release of the active ingredient from the dosage form over a time period of 45 minutes or more. This period starts usually with the administration of the dosage form, or with the start of in-vitro dissolution test (the moment the dosage form is brought into the dissolution medium). Preferably, the sustained release is achieved over a time period up to 16 hours and more. The dosage form combines one or more active ingredients with a mixture of hydrophilic polymer carriers resulting in a gel forming matrix composition. The above range includes all values and subranges therebetween, including 1, 2, 3, 4, 5, 6, 7, 8, 9, 10, 11, 12, 13, 14, 15, 16, 17, 18, 19, 20, 21, 22, 23 and 24 hours.

Preferably, the release profile is linear over the desired time, with little or no initial burst and with little or no lag time.

By the term substantially ion-strength independent is meant that the release rate profile of the active ingredient is not significantly changed (according to General Chapter 711: Physical tests and Determinations in USP 24 (±10% of label claim)) when the ion strength (I) is varied between 0.05 and 0.45 mol/L. The ion strength (I) is defined as I=1/2Σczᵢ² in which c is the concentration of the different ions in the solution and zᵢ their respective charge number (Handbook of Chemistry and Physics 71^{st} edition, David R. Lide ed., page 2-18, Boston, CRC Press Inc.; 1990-1991). The entire contents of each of the above references is hereby incorporated by reference.

Preferably the present invention achieves a sustained release, which is substantially independent from the ion-strength of the dissolution medium, which is normally the gastrointestinal fluid even when the composition is not coated.

Preferably, the composition is a pharmaceutical hydrophilic gel forming matrix composition having a extended release of one or more active ingredients upon exposure to gastrointestinal fluids, wherein the release is substantially ion-strength independent.

Although a coating is not essential to achieve the independency of the ion-strength, the composition may be optionally coated with one or more coating materials in order to achieve another desired effect, such as masking of the taste or application of color. Suitable coating materials are known in the art and include for example HPMC, acrylic, ethylcellulose (see Graham Cole ed., Pharmaceutical Coating Technology, London, Taylor & Francis Ltd.; 1995), the entire contents of which are hereby incorporated by reference.

The hydrophilic gel forming matrix has the form of tablets or of a multi-particulate dosage form and preferably contains a mixture of at least two hydrophilic high viscosity cellulose ethers.

Although the presence of a hydrophobic cellulose ether such as ethylcellulose will normally have no detrimental effect on the release properties of the present composition, it is preferable that the composition does not contain more than 20% by weight of hydrophobic cellulose ether(s), based on the weight of the gel forming polymers.

Cellulose ethers are well known in the art and are available in pharmaceutical grades and with different average molecular weights leading to different viscosities of a solution of these cellulose ethers. For present purposes, hydrophilic polymers may be characterized according to their viscosities, wherein low viscosity polymers include those having viscosities (in a 2% w/w aqueous solution at 25° C.) of less than about 1000 mPas, medium viscosity (about 1000 mPas to about 10,000 mPas and high viscosity (greater than about 10,000 mPas).

The above-mentioned viscosity ranges include all values and subranges therebetween, including 1, 5, 10, 20, 50, 70, 100, 125, 250, 500, 750, 800, 900, and 950 mPas (for low viscosity polymers); 1,000, 1,100, 2,000, 4,000, 5,000, 7,000, 9,000, 9,500, 9,750, and 10,000 mPas (for medium viscosity polymers); and 10,250, 10,500, 11,000, 12,000, 15,000, 19,000, 20,000, 50,000, 75,000, 90,000, 100,000, 125,000, 250,000, and 500,000 mPas (for high viscosity polymers).

Preferred hydrophilic hydroxypropyl methylcellulose polymers (HPMC's) which may be used in the present invention are available in different viscosity grades from Dow Chemical Co. under the brand name METHOCEL® and from Shin Etsu under the brand name METOLOSE®.

Preferred examples of low viscosity polymers include METHOCEL E5®, METHOCEL E-15LV®, METHOCEL E50LV®, METHOCEL K100LV®, and METHOCEL F50LV®, whose 2% aqueous solutions at 25° C. have viscosities of 5 mPas, 15 mPas, 50 mPas, 100 mPas, and 50 mPas respectively.

Preferred examples of medium viscosity HPMC's include METHOCEL E4M® and METHOCEL K4M®, whose 2% aqueous solutions at 25° C. have viscosities of 4,000 mPas.

Preferred examples of high viscosity HPMC's include METHOCEL K15M® and METHOCEL K100M®, whose 2% aqueous solutions at 25° C. have viscosities of 15,000 mPas and 100,000 mPas.

Preferred hydrophilic hydroxyethylcellulose polymers (HEC's) which may be used in the present invention are available in different viscosity grades from AQUALON® under the brand name NATROSOL® and from Amerchol Corporation under the brand name CELLOSIZE®.

Preferred examples of low viscosity polymers include NATROSOL L® and NATROSOL J®, whose 2% aqueous solutions at 25° C. have viscosities of 10 mPas and 20 mPas respectively.

Preferred examples of medium viscosity polymers include NATROSOL G® and NATROSOL K® whose 2% aqueous solutions at 25° C. have viscosities of 200 mPas and 1500 mPas, respectively.

Preferred examples of high viscosity polymers include NATROSOL M® and NATROSOL HH® whose 2% aqueous solutions have viscosities at 25° C. of 4,000 mPas and 90,000 mPas respectively.

In a preferred embodiment of the present invention the composition includes a mixture of a high or medium viscosity hydroxypropylmethylcellulose (HPMC) and a high or medium viscosity hydroxyethylcellulose (HEC), The ratio between the high or medium viscosity HPMC and the high or medium viscosity HEC is 1/0.5 to 1/1.5, preferably 1/0.85 to 1/1.2, more preferably 1/0.9 to 1/1.1, even more preferably is 1/0.95 to 1/1.05 and most preferred is 1/1. These ranges include all values and subranges therebetween, including 1/0.6, 1/0.7, 1/0.8, 1/0.87, 1/0.89, 1/0.91, 1/0.93, 1/0.95, 1/0.97, 1/0.99, 1/1.03, 1/1.07, 1/1.09, 1.13, 1/1.15, 1/1.17, 1/1.19, 1/1.2, 1/1.3, 1/1.4 and 1/1.5. Unless otherwise specified, the ratios given herein are weight ratios.

The composition optionally may include a low viscosity HPMC. In this case the ratio between high or medium viscosity HPMC and low viscosity HPMC is in the range between 1/0.01 and 1/0.2 and preferably is between 1/0.01 and 1/0.1 and even more preferably is between 1/0.02 and 1/0.05. These ranges include all values and subranges therebetween, including 1/0.03., 1/0.05, 1/0.07, 1/0.09, 1/0.11, 1/0.13, 1/0.15, 1/0.17 and 1/0.19.

It has surprisingly been found that the present compositions are particularly suitable for preparing tablets that have a release rate, which is independent of the ion-strength in the normal gastro-intestinal fluid range. The normal range is between 0.17 and 0.35 mol/L.

Apart from its independence from the ion-strength, the release of the composition is also substantially independent from the pH in the range between pH=1.3 and pH=7.4, which range includes all values and subranges therebetween, including 1.3, 1.4, 1.6, 1.8, 2.0, 2.5, 3.0, 3.5, 4.0, 4.5, 5.0, 5.5, 6.0, 6.5, 6.6, 6.7, 6.8, 6.9, 7.0, 7.1, 7.2, 7.3 and 7.4. This means that the release rate of active ingredient is not influenced by the pH in those cases where the active ingredient release is not limited by the solubility of the active ingredient, i.e., that the differences in release values (in %) at a given point in time are less than 20% of the label claim (see Chapter Dissolution Specifications (pages 1080-81) in FIP Guidelines for Dissolution Testing of Solid Oral Products (Final Draft, 1995), Drug Information Journal 1996, Vol 30, 1071-84) within the whole pH range between 1.3 and 7.4, the entire contents of which are hereby incorporated by reference.

The composition may preferably include one or more flow regulating agents such as colloidal silica. Colloidal silica may be present in an amount of 0.01-5%, which range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 3.5, 4, 4.1, 4.3, 4.5, 4.7 and 4.9%.

The composition may preferably include one or more lubricating agents such as talcum, sodium stearyl fumarate or magnesium stearate. Mixtures are possible. The lubricating agent may be present in an amount of 0.01-5%, which range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 3.5, 4, 4.1, 4.3, 4.5, 4.7 and 4.9%.

The hydrophilic celluloses in the complete composition are preferably present in a total amount ranging from 15% to 99.5% by weight, based on the weight of the tablet, which range includes all values and subranges therebetween, including 16, 17, 18, 19, 20, 25, 30, 35, 40, 45, 50, 55, 60, 65, 70, 75, 80, 85, 90 and 95%. Unless otherwise specified, percents are given in weight percent, based on the total weight of the tablet or composition.

The amount of active ingredient ranges from 0.1% to 80% by weight, which range includes all values and subranges therebetween, including 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75 and 80%.

The amount of glidant if present is between 0.01% and 5%, which range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 3.5, 4, 4.1, 4.3, 4.5, 4.7 and 4.9%, and is most preferably about 0.2%.

The amount of lubricant if present is preferably between 0.01% and 5%, which range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 3.5, 4, 4.1, 4.3, 4.5, 4.7 and 4.9%, and is preferably about 0.4%.

The composition may optionally include one or more pigments or pigment blends in a total amount of 0.01% and 10%, which range includes all values and subranges therebetween, including 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, and 9%. Typical pigment blends are commercially available, e.g. from COLORCON® as Opadry®.

The composition preferably meets the normal physical and pharmaceutical requirements in the art. Preferably, the powder form of the composition exhibits good flowing properties during tabletting, the compressed tablet form of the composition exhibits a crushing strength of at least 30 N, a friability below 1% at a compression force between 10 and 40 kN. In addition, the composition is preferably uniform and sufficiently stable. The composition can be prepared using normal composition procedures and equipment, so that no large investments are necessary.

The present invention also relates to a method of preparing the composition, wherein (1) a core is compressed of a mixture including one or more active ingredients and a mixture of at least two hydrophilic high or medium viscosity cellulose ethers yielding a substantially ion-strength independent and extended substantially zero-order release of active ingredients; and (2) the core is optionally coated.

The active ingredient, HPMC, HEC, optionally pigment blend and optionally glidant are mixed in a suitable mixer to form a powder mixture. This powder mixture is optionally blended with, for example, sodium stearyl fumarate in a suitable mixer.

The active ingredient is preferably added in the form of a pregranulate to the powder mixture for compression. Alternatively, the powder mixture for tabletting may be produced by a mixing procedure that is followed by a (wet or dry) granulation process.

Pregranulation preferably includes mixing a low-viscosity HPMC, pigment blend and active ingredient, spraying the mixture with one or more solvent(s), such as water, ether, benzene, methanol, ethanol, ethyl acetate, acetonitrile, and/or mixtures thereof, preferably water, while mixing and granulating, drying the obtained granulate (first granulate) until a residual solvent or water content of less than 1% is obtained. Then, a medium viscosity HPMC, an HEC, optionally pigment blend and optionally colloidal silica are screened and mixed to obtain a second granulate; and the first and second granulates are mixed together; sodium stearyl fumarate is optionally added to the mixed first and second granulates, and mixed; and then the whole is tableted.

Alternatively, a dry-mixing procedure may be used, which avoids pregranulation and which includes first mixing the active ingredient, low-viscosity HPMC, medium viscosity HPMC, HEC, optionally pigment blend, and optionally colloidal silica to obtain a first mixture; then optionally contacting the first mixture with sodium stearyl fumarate and mixing; and then the whole is tableted.

In a one-step dry-mixing procedure, all the ingredients are mixed for 2-10 minutes, which range includes 3, 4, 5, 6, 7, 8, and 9 minutes.

In a three-step dry-mixing procedure, a medium viscosity HPMC, active ingredient, and pigment blend are mixed to obtain a first mixture; then the HEC is added thereto and mixed to obtain a second mixture; then the low-viscosity HPMC and colloidal silica are added thereto and mixed to obtain a third mixture; and finally sodium stearyl fumarate is added and mixed; and then the whole is tableted. In the three-step procedure, the mixing times to obtain the first, second and third mixtures are each about the same, and the mixing time for the sodium stearyl fumarate is about ¼of the other individual times. The first, second and third mixing times may each range from 0.25-4 minutes, which range includes 0.5, 0.75, 1, 1.5, 2, 2.5, 3 and 3.5 minutes as appropriate.

The mixture of ingredients is compressed into tablets with commercial available equipment (e.g., a COURTOYE® R0) using flow regulating agents like colloidal silica and lubricating agents like talcum, sodium stearyl fumarate or magnesium stearate. The flow regulating and lubricating agent may be added to improve powder flow properties and to prevent sticking of powder to the dye walls or the punches.

One preferred composition includes 46.425% active hydroxypropylmethylcellulose K4M, 5.00% hydroxypropylmethylcellulose E5, 46.425% hydroxyethylcellulose HX 250, 1.45% active ingredient, 0.10% HPMC-based pigment blend, 0.20% colloidal silica V, and 0.40% sodium stearyl fumarate. Percents are by weight unless otherwise specified.

Preferred polymers, polymer formulations, and methods are disclosed in WO 02/26214, the entire contents of which are hereby incorporated by reference.

The pharmaceutically acceptable acid addition salts of the active ingredient of the invention include those in which the counter-ion does not contribute significantly to the toxicity or pharmacological activity of the salt and, as such, they are the pharmacological equivalents of the active ingredient base. They are generally preferred for medical usage. In some instances, they have physical properties which makes them more desirable for pharmaceutical formulation such as solubility, lack of hygroscopicity, compressibility with respect to tablet formation and compatibility with other ingredients with which the substance may be used for pharmaceutical purposes. The salts are routinely made by admixture of the base with the selected acid preferably by contact in solution employing an excess of commonly used inert solvents such as water, ether, benzene, methanol, ethanol, ethyl acetate, acetonitrile, and mixtures thereof. They may also be made by metathesis or treatment with an ion exchange resin under conditions in which the anion of one salt of the active ingredient is replaced by another anion under conditions which allow for separation of the desired species such as by precipitation from solution or extraction into a solvent, or elution from or retention on an ion exchange resin. The salt may be made from pharmacologically acceptable organic and inorganic acids such as hydrochloric, hydrobromic, sulfonic, sulfuric, phosphoric, nitric, maleic, fumaric, benzoic, ascorbic, pamoic, succinic, methanesulfonic, acetic, propionic, tartaric, citric, lactic, malic, mandelic, cinnamic, palmitic, itaconic, benzenesulfonic, hydroiodic, mucic, isethionic, heptanoic, and others. Mixtures of salts are possible.

The active ingredient may be used in one or more forms such as pharmaceutically acceptable acid addition salts, prodrugs, solvates, polymorphs, hydrates, anhydrates, crystalline forms, amorphous forms, stereoisomers as well as optical isomers, e.g. mixtures of enantiomers as well as individual enantiomers and diasteromers, which may arise as a consequence of structural asymmetry. Separation of the individual isomers is accomplished by application of various methods which are well known to practitioners in the art.

The dosage and dosage regimen is preferably adjusted utilizing sound professional judgment and considering the age, weight and condition of the recipient, the route of administration, and the nature and gravity of the illness. Preferably, the daily dose of active ingredient will be from about 0.05 to about 50 mg active ingredient/kg body weight when administered orally. Each of the above ranges includes all values and subranges therebetween, including 0.05, 0.06, 0.07, 0.08, 0.09, 0.1, 0.2, 0.3, 0.4, 0.5, 0.6, 0.7, 0.8, 0.9, 1,2,3,4, 5, 6, 7, 8, 9, 10, 13, 15, 17, 19, 20, 25, 30, 35, 40, 45, 46, 47, 48, 49 and 50 mg/kg as appropriate. In some instances, a sufficient therapeutic effect can be obtained at lower doses while in others, larger doses will be required. Though less preferred, the composition may also be administered nasally, rectally, via implant, and parenterally (i.e. intramuscular, intravenous and subcutaneous). In accordance with good clinical practice, it is preferred to administer the instant compounds at a concentration level that will produce the desirable effects without causing any harmful or untoward side effects.

The compounds of the present invention may be administered as individual therapeutic or preventive agents or as mixtures with other therapeutic or preventive agents. Therapeutically, they are generally given as pharmaceutical compositions comprised of an effective amount of active ingredient or a pharmaceutically acceptable salt thereof and a pharmaceutically acceptable carrier. Pharmaceutical compositions which provide from about 1 to 500 mg of active ingredient per unit dose are preferred, which range includes all values and subranges therebetween, including 2, 3, 5, 7, 9, 10, 20, 30, 40, 50, 75, 100, 150, 200, 250, 300, 400, 450 and 497 mg per unit dose.

Preferably, the subject is a human. More preferably, the subject is a human in need of prevention and/or treatment of at least one selected from the group including social phobia, general anxiety disorder, major depression, atypical depression, schizophrenia, psychotic disorder, and combinations thereof. The determination of the need for such prevention and/or treatment is within the skill level of the ordinary practitioner, such as a physician.

Preferred active ingredients, formula I, formula II, adatanserin and abaperidone, are described in more detail below.

Compound of Formula I

One active ingredient includes a compound having the formula (I):

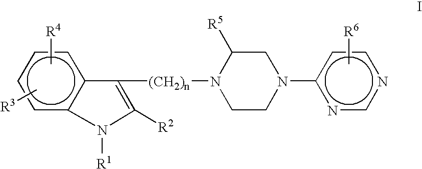

wherein R¹ is selected from the group including hydrogen, lower alkyl, and aryl-lower alkyl, e.g. benzyl. With respect to formula I, the descriptive term “lower” is used to denote an organic radical containing from 1 to 4 carbon atoms; aryl means phenyl or R³-substituted phenyl; R² and R⁵ are independently selected from hydrogen and lower alkyl; R³ and R⁴ are independently selected from among hydrogen, lower alkyl, lower alkoxy, lower alkylthio, carboxamide, halogen and trifluoromethyl; R⁶ is lower alkoxy and n is the integer 2 or 3. Preferred classes of formula I compounds are those wherein R³ is 5-fluoro- and wherein R⁵ is 5-methoxy.

The compounds of formula I and methods for making and for using are disclosed in U.S. Pat. No. 5,077,293, the entire contents of which are hereby incorporated by reference.

With respect to the compound of formula I, halogen denotes fluorine, chlorine, bromine and iodine; with the term “lower alkyl” referring to both straight and branched chain carbon radicals of from to 4 carbon atoms inclusive. Illustrative of these radicals are carbon chains which can be methyl, ethyl, propyl, isopropyl, 1-butyl, 1-methylpropyl and 2-methylpropyl. Carboxamide intends a —C(═O)NH₂ radical.

One preferred embodiment of the instant invention provides a method for treating a mammal afflicted with depression or chronic pain which includes administering to said mammal a therapeutically effective amount of a compound of formula I or a pharmaceutically acceptable acid addition salt thereof.

Another preferred embodiment of the invention provides a method for treating a mammal afflicted with social phobia, which includes administering to said mammal a therapeutically effective amount of a compound of formula I or a pharmaceutically acceptable acid addition salt thereof.

Compounds having the formula (I) inhibits the re-uptake of endogenous serotonin. These compounds are particularly suitable in the following disorders: obsessive-compulsive disorder, feeding disorder, anxiety disorder, panic disorder, social disorder and social phobia. In addition, they inhibit norepinephrine re-uptake, and blockade of endogenous norepinephrine re-uptake is also a mechanism through which it is believed that various antidepressant agents exert their therapeutic effect (see: “Antidepressants: Neurochemical, Behavioral, and Clinical Perspectives”, edited by S. J. Enna, J. B. Malick and E. Richardson, (1981), Raven Press, New York, pp. 1-12), the entire contents of which are hereby incorporated by reference.

Determination of endogenous monoaminergic re-uptake inhibition values both for serotonin and norepinephrine may be accomplished using test methods described by P. Skolnick, et al., Br. J. Pharmacology (1985), 86, pp. 637-644 (the entire contents of which are hereby incorporated by reference) with only minor modifications. In vitro IC₅₀ (nM) test values may be determined for representative compounds of formula I based on their inhibition of synaptosomal reuptake of tritiated serotonin. Test data IC₅₀ values lower than 500 nM are considered to reflect activity as an inhibitor of serotonin reuptake. Compounds with IC₅₀ values lower than 100 nM are preferred.

The administration and dosage regimen of compounds of formula I may optionally be carried out in the same manner as for the compound fluoxetine, cf: Schatzberg, et al., J. Clin. Psychopharmacology 7/6 Suppl. (1987) pp. 4451-4495, the entire contents of which are hereby incorporated by reference.

Preferred compounds of formula I include those wherein R¹ is selected from the group including hydrogen, lower alkyl and lower alkyl meaning C₁₋₄; R² and R⁵ are independently selected from the group including hydrogen and lower alkyl; R³ and R⁴ are independently selected from the group includiong hydrogen, lower alkyl, lower alkoxy, lower alkylthio, carboxamide, halogen and trifluoromethyl; R⁶ is lower alkoxy; and n is the integer 2 or 3.

Another preferred compound of formula I is one in which R⁴ is 5-fluoro.

Another preferred compound of formula I is one in which R⁶ is 5-methoxy.

Preferred examples of the compound of formula I include 1-(3-(1H-indol-3-yl)propyl)-4-(5-methoxy-4-pyrimidinyl)piperazine; 1-(2-(5-fluoro-1H-indol-3-yl)ethyl)-4-(5-methoxy-4-pyrimidinyl)piperazine; 1-(2-(5-fluoro-1H-indol-3-yl)ethyl)-4-(5-methoxy-4-pyrimidinyl)-2-methylpiperazine; 1-(3-(5-fluoro-1H-indol-3-yl)propyl)-4-(5-methoxy-4-pyrimidinyl)-2-methylpiperazine; 1-(3-(5-fluoro-1H-indol-3-yl)propyl)-4-(5-methoxy-4-pyrimidinyl)piperazine; 4-(5-methoxy-4-pyrimidinyl)-1-(3-(1-methyl-1H-indol-3-yl)propyl)-2-methylpiperazine; 1-(3-(6-fluoro-1H-indol-3-yl)propyl)-4-(5-methoxy-4-pyrimidinyl)-2-methylpiperazine; 1-(3-(5-chloro-1H-indol-3-yl)propyl)-4-(5-methoxy-4-pyrimidinyl)-2-methylpiperazine; 1-(3-(1H-indol-3-yl)propyl)-4-(6-methoxy-4-pyrimidinyl)piperazine; 1-(3-(1H-indol-3-yl)propyl)-4-(6-methoxy-4-pyrimidinyl)-2-methylpiperazine; 1-(3-(5-chloro-1H-indol-3-yl)propyl)-4-(5-methoxy-4-pyrimidinyl)piperazine; 1-(3-(5,6-difluoro-1H-indol-3-yl)propyl)-4-(5-methoxy-4-pyrimidinyl)-2-methylpiperazine; 1-(3-(5,6-difluoro-1-indol-3-yl)propyl)-4-(5-methoxy-4-pyrimidinyl)piperazine; 2-ethyl-1-(3-(1H-indol-3-yl)propyl)-4-(5-methoxy-4-pyrimidinyl)piperazine; 4-(5-ethoxy-4-pyrimidinyl)-1-(3-(1H-indol-3-yl)propyl)piperazine; 4-(5-ethoxy-4-pyrimidinyl)-1-(3-(1H-indol-3-yl)propyl)-2-methylpiperazine; and 1H-indole, 5-fluoro-3-(3-(4-(5-methoxy-4-pyrimidinyl)-1-piperazinyl)propyl)-,dihydrochloride.

One preferred compound of formula I has the following structure:

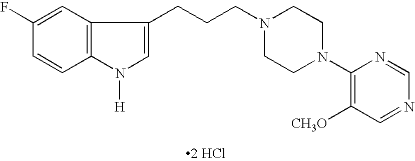

One embodiment of the present invention provides a method for treating or preventing at least one selected from the group including social phobia, general anxiety disorder, major depression, atypical depression, schizophrenia, psychotic disorder, and combinations thereof which includes administering the composition of the invention including the compound of formula I to a subject.

A preferred embodiment of the present invention provides a method for treating or preventing at least one selected from the group including major depression, atypical depression, and combinations thereof which includes administering the composition of the invention including the compound of formula I to a subject.

Compound of Formula II

Another active ingredient includes a compound having formula II:

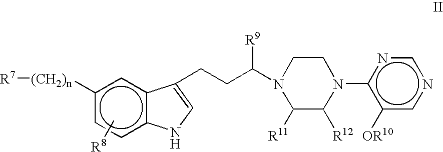

wherein R⁷ is selected from the group including hydrogen; halogen lower alkyl; lower alkoxy; phenyl-lower alkoxy, with the phenyl ring unsubstituted or bearing a lower alkyl substituent; amino; cyano; hydroxy; nitro; —OCH₂CN; —OCH₂CONR¹³R¹⁴; —SO₂NR¹³R¹⁴; —O₂CR¹⁵; —SO₂R¹⁵; —O₂CNR¹³R¹⁴; —COR¹⁴; —CO₂R¹⁵; —CONR¹³R¹⁴; —NR¹³CO₂R¹⁵; —NR¹³COR¹⁴; —NR¹³SO₂R¹⁵; and

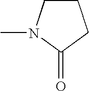

R⁸ is selected from hydrogen, halogen, lower alkyl, lower alkoxy and —CO₂R¹⁵.

R⁹, R¹¹, R¹² and R¹³ are independently selected from the group including hydrogen and lower alkyl. R⁹ however cannot be hydrogen when the R⁷—(CH₂)ₙ moiety is hydrogen, halogen, lower alkyl, lower alkoxy or —CONH₂ and R⁸ is hydrogen, halogen or lower alkyl.

R¹⁰ is lower alkyl.

R¹⁴ is selected from the group including hydrogen, lower alkyl, R¹³-substituted phenyl-lower alkyl and trifluoromethyl.

R¹⁵ is selected from lower alkyl and R¹³-substituted phenyl-lower alkyl.

The symbol n is zero or the integers 1 or 2.

The compounds of formula II and methods for making and using are disclosed in U.S. Pat. No. 5,300,506, the entire contents of which are hereby incorporated by reference.

In the context of formula II, the term “lower alkyl” refers to both straight and branched chain carbon radicals of from 1 to 4 carbon atoms inclusive. Illustrative of these radicals are carbon chains which can be methyl, ethyl, propyl, isopropyl, 1-butyl, 1-methylpropyl 2-methylpropyl.

Preferred compounds of formula II are those wherein R⁷ is a polar substituent attached to the 5-position of the indole ring.

One embodiment of the present invention provides a method for treating a vascular headache sufferer which includes systemic administration to the sufferer of a therapeutically effective amount of a compound of formula II or a pharmaceutically acceptable salt and/or solvate thereof.

Another preferred embodiment of the invention provides a method for treating a mammal afflicted with social phobia, which includes administering to said mammal a therapeutically effective amount of a compound of formula II or a pharmaceutically acceptable acid addition salt thereof.

Preferred compounds of formula II includes those wherein R⁹ can be hydrogen regardless of the nature of R⁷. These compounds include those wherein R⁷ is selected from the group including hydrogen, halogen, lower alkyl, lower alkoxy, R¹³-substituted phenyl-lower alkoxy, amino, cyano, hydroxy, nitro, —OCH₂ CN, —OCH₂CONR¹³R¹⁴, —SO₂NR¹³R¹⁴, —O₂CR¹⁵, —SO₂R¹⁵, —O₂CNR¹³R¹⁴, —COR₂R¹⁵, —CONR¹³R¹⁴, NR¹³CO₂R¹⁵, —NR¹³COR¹⁴, —NR¹³SO₂R¹⁵ and 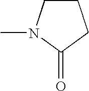
;
R⁸ is selected from the group including hydrogen, halogen, lower alkyl, lower alkoxy and —CO₂R¹⁵; R⁹, R¹¹, R¹² and R¹³ are independently selected from the group including hydrogen and lower alkyl; R¹⁰ is lower alkyl; R¹⁴ is selected from the group including hydrogen, lower alkyl, R¹³-phenyl-lower alkyl and trifluoromethyl; R¹⁵ is selected from the group including lower alkyl and R¹³-phenyl-lower alkyl; and n is zero or the integers 1 or 2.

The compounds of formula II demonstrates potent affinity and agonist activity at the 5-HT_{ID} site. Formula II compounds have potencies wherein IC₅₀ values of these compounds are less than 100 nmolar. Preferred compounds have IC₅₀ values below 10 nmolar.

Determination of 5-HT_{ID} binding properties may be accomplished employing methodology such as that described by Heuring and Peroutka, J. Neurosci., 7(3), 1987, 894-903 (the entire contents of which are hereby incorporated by reference); with only minor modifications. In vitro IC₅₀ (nM) test values may be determined for the compounds of formula II employing tritiated serotonin.

The administration and dosage regimen of compounds of Formula II may optionally be carried out in the same manner as for the compound sumatriptan, cf: Oxford, GB 2,1 62,522A, the entire contents of which are hereby incorporated by reference.

Preferred compounds of formula II include those in which R⁷ is selected from the group including hydrogen, halogen, lower alkyl, lower alkoxy, R¹³-substituted phenyl-lower alkoxy, amino, cyano, hydroxy, nitro, —OCH₂CN, —OCH₂CONR¹³R¹⁴, —SO₂NR¹³R¹⁴, —O₂CR¹⁵, —SO₂ R¹⁵, —O₂CNR¹³R¹⁴, —COR¹⁴, —CO₂R¹⁵, —CONR¹³R¹⁴, —NR¹³CO₂R¹⁵, —NR¹³COR¹⁴, —NR¹³SO₂R¹⁵ and 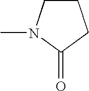
R⁸ is selected from the group including hydrogen, lower alkyl, lower alkoxy, —CO₂R¹⁵ and halogen;

R⁹ is independently selected from the group including hydrogen and lower alkyl, with the proviso that R⁹ cannot be hydrogen when the R⁷—(CH₂)ₙ moiety is hydrogen, lower alkyl, lower alkoxy, halogen or —CONH₂ and R⁸ is hydrogen, lower alkyl and halogen;

R¹¹, R¹² and R¹³ are independently selected from the group including hydrogen and lower alkyl;

R¹⁰ is lower alkyl;

R¹⁴ is selected from the group including hydrogen, lower alkyl, R¹³-phenyl-lower alkyl and trifluoromethyl;

R¹⁵ is selected from the group including lower alkyl and R¹³-phenyl-lower alkyl; and

n is zero or the integers 1 or 2.

Another preferred compound of formula II is one in which R⁷ is selected from the group including —SO₂NR¹³R¹⁴ and —SO₂R¹⁵.

Another preferred compound of formula II is one in which R⁷ is selected from the group including hydrogen, halogen, lower alkyl, lower alkoxy, R¹³-phenyl-lower alkoxy, amino, cyano, hydroxy, and nitro.

Another preferred compound of formula II is one in which R⁷ is selected from the group including —OCH₂CN, —OCH₂CONR¹³R¹⁴, —O₂CR¹⁵ and —O₂CNR¹³R¹⁴.

Another preferred compound of formula II is one in which R⁷ is selected from the group including —COR¹⁴, —CO₂R¹⁵ and CONR¹³R¹⁴.

Another preferred compound of formula II is one in which R⁷ is selected from the group including —NR¹³SO₂R¹⁵, —NR¹³CO₂R¹⁵, —NR¹³COR¹⁴ and

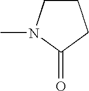

Another preferred compound of formula II is one in which R⁷ is —SO₂NR¹³R¹⁴, and which is selected from the group including 4-(5-methoxy-4-pyrimidinyl)-1-(3-(5-(((methylamino)sulfonyl)methyl)-1H-ind ol-3-yl)propyl)piperazine; 4-(5-methoxy-4-pyrimidinyl)-1-(3-(5-(((methylamino)sulfonyl)methyl)-1H-indol-3-yl)propyl)-3-methylpiperazine; and 1-(3-(5-(((dimethylamino)sulfonyl)methyl)-1H-indol-3-yl)propyl)-4-(5-methoxy-4-pyrimidinyl)piperazine; and 1-(3-(5-((aminosulfonyl)methyl)-1H-indol-3-yl)propyl)-4-(5-methoxy-4-pyrimidinyl)piperazine.

Another preferred compound of formula II is one in which R⁷ is —SO₂ R¹⁵, and which is selected from the group including 4-(5-methoxy-4-pyrimidinyl)-1-(3-(5-(2-(methylsulfonyl)methyl)-1H-indol-3-yl)propyl)-3-methylpiperazine; 4-(5-methoxy-4-pyrimidinyl)-1-(3-(5-(2-(methylsulfonyl)methyl)-1H-indol-3-yl)propyl)-3-methylpiperazine; 4-(5-methoxy-4-pyrimidinyl-1-(3-(5-(2-(methylsulfonyl)ethyl)-1H-indol-3-yl)propyl)piperazine; and 4-(5-methoxy-4-pyrimidinyl)-1-(3-(5-(2-(methylsulfonyl)ethyl)-1H-indol-3-yl)propyl)-3-methylpiperazine.

Other preferred compounds of formula II are those selected from the group including 1-(3-(5-cyano-1H-indol-3-yl)propyl)-4-(5-methoxy-4-pyrimidinyl)piperazine; 1-(3-(5-cyano-1H-indol-3-yl)propyl)-4-(5-methoxy-4-pyrimidinyl)-3-methylpiperazine; 3-(3-(4-(5-methoxy-4-pyrimidinyl)-1-piperazinyl)-propyl)-5-nitroindole; 3-(3-(4-(5-methoxy-4-pyrimidinyl)-1-piperazinyl)propyl)-5-aminoindole; 1-(3-(1H-indol-3-yl)-3-methylpropyl)-4-(5-methoxy-4-pyrimidinyl)piperazine; 1-(3-(1H-indol-3-yl)propyl)-4-(5-methoxy-4-pyrimidinyl)-3-methylpiperazine; 1-(3-(5-hydroxy-1H-indol-3-yl)propyl)-4-(5-methoxy-4-pyrimidinyl)piperazine; 3-(3-(4-(5-methoxy-4-pyrimidinyl)-1-piperazinyl)-3-ethylpropyl)indole; 1-(3-(5-phenylmethoxy-1H-indol-3-yl)propyl)-4-(5-methoxy-4-pyrimidinyl)piperazine; 3-(3-(4-(5-methoxy-4-pyrimidinyl)-1-piperazinyl)propyl)-5-fluoro-6-acetoxyindole; and 3-(3-(4-(5-methoxy-4-pyrimidinyl-1-piperazinyl)propyl)-5-fluoro-6-methoxyindole.

Other preferred compounds of formula II are those selected from the group including 3-(3-(4-(5-methoxy-4-pyrimidinyl)-1-piperazinyl)propyl)-5-(cyanomethyl)oxyindole and 3-(3-(4-(5-methoxy-4-pyrimidinyl)-1-piperazinyl)propyl)-5-(carboxamidomethyl)oxyindole.

Other preferred compounds of formula II are those selected from the group including 1-(3-(5-aminocarbonyl-1H-indol-3-yl)propyl)-4-(5-methoxy-4-pyrimidinyl)-3-methylpiperazine; 1-(3-(5-acetyl-1H-indol-3-yl)propyl)-4-(5-methoxy-4-pyrimidinyl)piperazine; 1-(3-(5-((aminocarbonyl) methyl)-1H-indol-3-yl)propyl)-4-(5-methoxy-4-pyrimidinyl)piperazine; 4-(5-methoxy-4-pyrimidinyl)-1-(3-(5-(((phenylmethylamino)carbonyl)methyl)-1H-indol-3-yl)propyl)piperazine; 1-(3-(5-(ethoxycarbonyl)-1H-indol-3-yl)propyl)-4-(5-methoxy-4-pyrimidinyl) piperazine; 1-(3-(5-(methoxycarbonyl)-1H-indol-3-yl)propyl)-4-(5-methoxy-4-pyrimidinyl) piperazine; 4-(5-methoxy-4-pyrimidinyl)-1-(3-(5-((methylamino) carbonyl)-1H-indol-3-yl)propyl)piperazine; 1-(3-(5-((ethylamino)oarbonyl)-1H-indol-3-yl)propyl)-4-(5-methoxy-4-pyrimidinyl)piperazine; 1-(3-(5-((n-butylamino)carbonyl)-1H-indol-3-yl)propyl)-4-(5-methoxy-4-pyrimidinyl)piperazine; 4-(5-methoxy-4-pyrimidinyl)-1-(3-(5-((1-(2-phenylethyl) amino)carbonyl)-1H-indol-3-yl)propyl)piperazine; and 4-(5-methoxy-4-pyrimidinyl)-1-(3-(5-(((phenylmethyl)amino)carbonyl)-1H-indol-3-yl)propyl)piperazine.

Other preferred compounds of formula II are those in which R⁷ is —NR¹³SO₂R¹⁵, and which are selected from the group including 4-(5-methoxy-4-pyrimidinyl)-1-(3-(5-(methyl(methylsulfonyl)amino)-1-H-indo 1-3-yl)propyl)-2-methylpiperazine; 4-(5-methoxy-4-pyrimidinyl)-1-(3-(5-((methylsulfonyl)amino)-1-H-indol-3-yl)propyl)-2-methylpiperazine; 1-((5-(((ethylsulfonyl)amino)-methyl)-1H-indol-3-3-yl)propyl)-4-(5-methoxy-4-pyrimidinyl)-3-methylpiperazine; 1-(3-(5-((ethylsulfonyl)amino)-1H-indol-3-yl)propyl)-4-(5-methoxy-4-pyrimidinyl)-2-methylpiperazine; 1-((5-((ethylsulfonyl)amino)methyl)-1H-indol-3-yl)propyl)-4-(5-methoxy-4-pyrimidinyl)-2-methylpiperazine; 1-((5-(((ethylsulfonyl)amino)methyl)-1H-indol-3-yl)propyl)-4-(5-methoxy-4-pyrimidinyl)piperazine; 4-(5-methoxy-4-pyrimidinyl)-1-(3-(5-(methyl(ethylsulfonyl)amino)-1H-indol-3-yl)propyl)-2-methylpiperazine; 4-(5-methoxy-4-pyrimidinyl)-1-((5-(((methylsulfonyl)amino)methyl)-1H-indol -3-yl)propyl)piperazine; 1-((5-(((methylsulfonyl)amino)methyl)-1H-indol-3-yl)propyl)-4-(5-methoxy-4-pyrimidinyl)-3-methylpiperazine; 1-((5-((ethyl(methylsulfonyl)amino)methyl)-1H-indol-3-yl)propyl)-4-(5-methoxy-4-pyrimidinyl) piperazine; 4-(5-methoxy-4-pyrimidinyl)-1-((5-((methyl(ethylsulfonyl)amino)methyl)-1H-indol-3-yl)propyl)piperazine; 4-(5-methoxy-4-pyrimidinyl)-1-((5-((phenylmethyl(ethylsulfonyl)amino)-methyl)-1H-indol-3-yl)propyl)piperazine; 4-(5-methoxy-4-pyrimidinyl)-1-((5-((phenylmethyl(methylsulfonyl)amino)methyl)-1H-indol-3-yl) propyl)piperazine; 4-(5-methoxy-4-pyrimidinyl)-1-((5-((methyl(methylsulfonyl)amino)methyl)-1H-indol-3-yl)propyl)piperazine; 4-(5-methoxy-4-pyrimidinyl)-1-(3-(5-(methyl(trifluoromethylsulfonyl)amino)-1H-indol-3-yl)propyl)piperazine; 1-(3-(5-methylsulfonyl)amino-1H-indol-3-yl)propyl)-4-(5-methoxy-4-pyrimidinyl) piperazine; 1-(3-(5-(methylsulfonyl)amino-1H-indol-3-yl)propyl)-4-(5-methoxy-4-pyrimidinyl)-3-methylpiperazine; 1-(3-(5-(ethylsulfonyl)amino-1H-indol-3-yl)propyl)-4-(5-methoxy-4-pyrimidinyl)piperazine; 1-(3-(5-(ethylsulfonyl)methylamino-1H-indol-3-yl)propyl)-4-(5-methoxy-4-pyrimidinyl) piperazine; 1-(3-(5-(ethylsulfonyl)amino-1H-indol-3-yl)propyl)-4-(5-methoxy-4-pyrimidinyl)-3-methylpiperazine; and 1-(3-(5-(((4-methylphenyl) sulfonyl)amino)-1H-indol-3-yl)propyl-4-(5-methoxy-4-pyrimidinyl)piperazine.

Other preferred compounds of formula II are those in which R⁷ is —NR¹³COR¹⁴, and which are selected from the group including 1-((5-(((acetyl)amino)methyl)-1H-indol-3-yl)propyl)-4-(5-methoxy-4-pyrimidinyl)piperazine; 1-(3-(5-formylamino-1H-indol-3-yl)propyl)-4-(5-methoxy-4-pyrimidinyl)piperazine; 1-(3-(5-acetylamino-1H-indol-3-yl)propyl)-4-(5-methoxy-4-pyrimidinyl)piperazine; and 1-(3-(5-((trifluoromethyl)carbonyl)amino-1H-indol-3-yl)propyl)-4-(5-methoxy-4-pyrimidinyl)piperazine.

Other preferred compounds of formula II are those selected from the group including 1-(3-(5-(((phenylmethoxy)carbonyl)amino)-1H-indol-3-yl)propyl)-4-(5-methoxy-4-pyrimidinyl)piperazine; 1-(3-(5-(((methoxy)carbonyl)amino)-1H-indol-3-yl)propyl)-4-(5-methoxy-4-pyrimidinyl)piperazine; and 1-(3-(5-(2-pyrrolidinon-1-yl)-1H-indol-3-yl)propyl)-4-(5-methoxy-4-pyrimidinyl)piperazine.

Other preferred compounds of formula II include 4-(5-methoxy-4-pyrimidinyl)-1-(3-(5-(((methylamino)sulfonyl)methyl)-1H-indol-3-yl)propyl)piperazine.

One embodiment of the present invention provides a method for treating and/or preventing at least one selected from the group including social phobia, general anxiety disorder, major depression, atypical depression, schizophrenia, psychotic disorder, and combinations thereof which includes administering the composition of the invention including the compound of formula II to a subject.

A preferred embodiment of the present invention provides a method for treating and/or preventing at least one selected from the group including major depression, atypical depression, and combinations thereof which includes administering the composition of the invention including the compound of formula II to a subject.

Adatanserin

Another active ingredient is the compound known generally as adatanserin, which has the following formula (III):

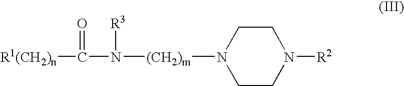

wherein R¹ is 1-adamantyl, 3-methyl-1-adamantyl, 3-noradamantyl, unsubstituted or substituted-2indolyl, 3-indolyl wherein the substituents are selected from the group including lower alkyl, lower alkoxy and halo; R² is unsubstituted or substituted phenyl, benzyl or pyridinyl, wherein the substituents are selected from the group including lower alkyl, lower alkoxy, trifluoromethyl and halo; R³ is H or lower alkyl of 1, 2 or 3 carbon atoms; n is the integer 0 or 1; and m is the integer of 2, 3, 4 or 5; or a pharmaceutically acceptable salt thereof.

A preferred compound has the following formula:

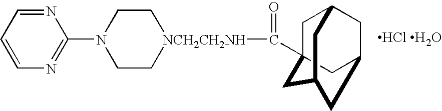

Other preferred compounds include N-(3-(4-(2-methoxyphenyl)-1-piperazinyl)propyl)tricyclo(3.3.1.1.^{3,7})decane-1-carboxamide; N-(2-(4-(2-pyrimidinyl)-1-piperazinyl)ethyl)tricyclo(3.3.1.1.^{3,7})decane-1-carboxamide; N-(2-(4-(2-methoxyphenyl)-1-piperazinyl)ethyl)tricyclo(3.3.1.1.^{3,7})decane-1-carboxamide; N-(2-(4-(3-chlorophenyl) 1-piperazinyl)ethyl)tricyclo(3.3.1.1.^{3,7})decane-1-carboxamide; N-(2-(4-(2-pyrimidinyl)-1-piperazinyl)ethyl)-2-benzofurancarboxamide; N-(3-(4-(2-methoxyphenyl)-1-piperazinyl)propyl)-2-benzofurancarboxamide; N-(3-(4-(2-methoxyphenyl)-1-piperazinyl)propyl)-1H-indole-2-carboxamide; N-(3-(4-(3-chlorophenyl)-1-piperazinyl)propyl)tricyclo(3.3.1.1.^{3,7})decane-1-carboxamide; N-(2-(4-(2-pyrimidinyl)-1-piperazinyl)ethyl)-3-methyltricyclo(3.3.1.1.^{3,7})decane-1-acetamide; N-(3-(4-(2-pyrimidinyl)-1-piperazinyl)propyl)tricyclo(3.3.1.1.^{3,7})decane-1-carboxamide; N-(2-(4-(3-chlorophenyl)-1-piperazinyl)ethyl)-3-methyltricyclo(3.3.1.1.^{3,7})decane-1-acetamide; N-(2-(4-(2-methoxyphenyl)-1-piperazinyl)ethyl)-3-methyltricyclo(3.3.1.1.^{3,7})decane-1-acetamide; N-(2-(4-(2-pyrimidinyl)-1-piperazinyl)ethyl)hexahydro-2,5-methanopentalene -3a(1H)-carboxamide; N-(2-(4-(2-methoxyphenyl)-1-piperazinyl)ethyl)hexahydro-2,5-methanopentalene-3a(1H)-carboxamide; N-(2-(4-(3-chlorophenyl)-1-piperazinyl)ethyl)hexahydro-2,5-methanopentalene -3a(1H)-carboxamide; N-(2-(4-(3-(trifluoromethyl)phenyl)-1-piperazinyl)ethyl)hexahydro-2,5-methanopentalene-3a(1H)-carboxamide; N-(3-(4-(2-pyrimidinyl)-1-piperazinyl)propyl)hexahydro-2,5-methanopentalene -3a(1H)-carboxamide; N-(3-(4-(2-methoxyphenyl)-1-piperazinyl)propyl)hexahydro-2,5-methanopentalene-3a(1H)-carboxamide; N-(3-(4-(3-chlorophenyl)-1-piperazinyl)propyl)hexahydro-2,5-methanopentalene-3a(1H)-carboxamide; and the pharmaceutically acceptable salts thereof.

Preferably, the compound having the chemical name, N-(2-(4-(2-pyridinyl)-1-piperazinyl)ethyl)tricyclo(3.3.1.^{3,7})decane-1-carboxamide and/or its hydrochloride monohydrate salt is used.

With respect to this compound, the term “lower alkyl” refers to moieties having 1 to 6 carbon atoms in the carbon chain, which includes those having 1, 2, 3, 4, 5 and 6 carbons. The term “alkoxy” refers to moieties having 1 to 6 carbon atoms, which includes those having 1, 2, 3, 4, 5 and 6 carbons. The term “halo” refers to fluoro, chloro and bromo.

Adatanserin and methods of making and using same are disclosed in U.S. Pat. No. 5,254,552, U.S. Pat. No. 5,278,160, U.S. Pat. No. 5,482,940, U.S. Pat. No. 5,380,725, U.S. Pat. No. 5,010,078, and U.S. Pat. No. 5,106,849, the entire contents of each of which being hereby incorporated by reference.

One embodiment of the present invention provides a method for treating and/or preventing at least one selected from the group including social phobia, general anxiety disorder, major depression, atypical depression, schizophrenia, psychotic disorder, and combinations thereof which includes administering the composition of the invention including adatanserin to a subject.

A preferred embodiment of the present invention provides a method for treating and/or preventing at least one selected from the group including social phobia, general anxiety disorder, major depression, and combinations thereof which includes administering the composition of the invention including adatanserin to a subject.

Abaperidone

Another active ingredient is the compound known generally as abaperidone, which has the following formula (IV):

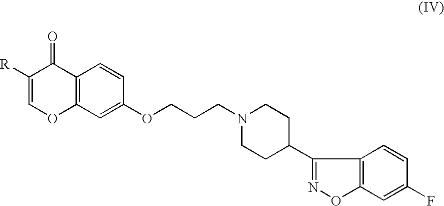

wherein R is hydrogen or alkyl having 1, 2, 3 or 4 carbon atoms, optionally substituted by hydroxyl, or a pharmaceutically acceptable addition salt thereof.

A preferred compound has the structure:

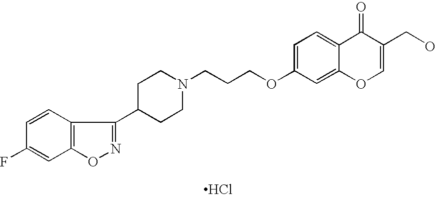

Other preferred compounds include 7-(3-(4-(6-fluoro-1,2-benzisoxazole-3-yl)piperidin-1-yl)propoxy)chromen-4-one; 7-(3-(4-(6-fluoro-1,2-benzisoxazole-3-yl)piperidin-1-yl)propoxy)-3-methyl-chromen-4-one; and 7-(3-(4-(6-fluoro-1,2-benzisoxazole-3-yl)piperidin-1-yl)propoxy)-3-(hydroxymethyl)-chromen-4-one; or a pharmaceutically acceptable addition salt thereof.

Abaperidone and methods of making and using same are disclosed in U.S. Pat. No. 5,736,558 and U.S. Pat. No. 6,348,605, the entire contents of each of which being hereby incorporated by reference.

One embodiment of the present invention provides a method for treating and/or preventing at least one selected from the group including social phobia, general anxiety disorder, major depression, atypical depression, schizophrenia, psychotic disorder, and combinations thereof which includes administering the composition of the invention including abaperidone to a subject.

A preferred embodiment of the present invention provides a method for treating and/or preventing at least one selected from the group including schizophrenia, psychotic disorder, and combinations thereof which includes administering the composition of the invention including abaperidone to a subject.

Each of the above-mentioned patents, applications and publications is incorporated herein by reference, the same as set forth at length.

Obviously, numerous modifications and variations of the present invention are possible in light of the above teachings. It is therefore to be understood that within the scope of the appended claims, the invention may be practiced otherwise than as specifically described herein.

1. A pharmaceutical composition, comprising:
at least one active ingredient selected from the group consisting of adatanserin, a compound having formula I, a compound having formula II, abaperidone, pharmaceutically acceptable salt thereof, prodrug thereof, and a mixture thereof;
at least one high or medium viscosity hydroxypropylmethylcellulose (HPMC);
at least one high or medium viscosity hydroxyethylcellulose (HEC);
wherein the HPMC and HEC are present in a HPMC/HEC weight ratio ranging from 1/0.5 to 1/1.5;
wherein the compound having the formula (I) is: 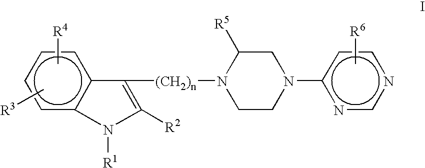
wherein R¹ is selected from the group consisting of hydrogen, lower alkyl and aryl-lower alkyl, R² and R⁵ are each independently selected from the group consisting of hydrogen and lower alkyl; R³ and R⁴ are each independently selected from the group consisting of hydrogen, lower alkyl, lower alkoxy, lower alkylthio, carboxamide, halogen and trifluoromethyl; R⁶ is lower alkoxy, and n is the integer 2 or 3;
and wherein the compound of formula (II) is: 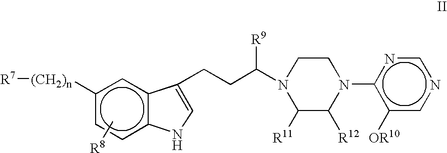
wherein R⁷ is selected from the group consisting of hydrogen, halogen, lower alkyl, lower alkoxy, phenyl-substituted lower alkoxy, said phenyl being unsubstituted or bearing a lower alkyl substituent, amino, cyano, hydroxy, nitro, —OCH₂CN; —OCH₂CONR¹³R¹⁴; —SO₂NR¹³R¹⁴; —O₂CR¹⁵; —SO₂R¹⁵; —O₂CNR¹³R¹⁴; —COR¹⁴; —CO₂R¹⁵; —CONR¹³R¹⁴; —NR¹³CO₂R¹⁵; —NR¹³COR¹⁴; —NR¹³SO₂R¹⁵; and 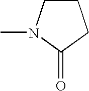
R⁸ is selected from the group consisting of hydrogen, halogen, lower alkyl, lower alkoxy and —CO₂R¹⁵;
R⁹, R¹¹, R¹² and R¹³ are each independently selected from the group consisting of hydrogen and lower alkyl with the proviso that R⁹ is not hydrogen when the R⁷—(CH₂)ₙ moiety is hydrogen, halogen, lower alkyl, lower alkoxy or —CONH₂ and R⁸ is hydrogen, halogen or lower alkyl;
R¹⁰ is lower alkyl;
R¹⁴ is selected from the group consisting of hydrogen, lower alkyl, R¹³-substituted phenyl-lower alkyl and trifluoromethyl;
R¹⁵ is selected from the group consisting of lower alkyl and R¹³-substituted phenyl-lower alkyl; and
n is 0, 1, or 2.

2. The composition according to claim 1, further comprising a coating.

3. The composition according to claim 1, further comprising at least one low viscosity HPMC.

4. The composition according to claim 1, further comprising at least one low viscosity HPMC, wherein the high or medium viscosity HPMC and the low viscosity HPMC are present in a high or medium viscosity HPMC/low viscosity HPMC weight ratio ranging from 1/0.01 to 1/0.2

5. The composition according to claim 1, further comprising silica or colloidal silica.

6. The composition according to claim 1, further comprising sodium stearyl fumarate.

7. The composition according to claim 1, wherein the active ingredient is adatanserin, pharmaceutically acceptable salt thereof, prodrug thereof, or a mixture thereof.

8. The composition according to claim 1, wherein the active ingredient is the compound of formula I, pharmaceutically acceptable salt thereof, prodrug thereof, or a mixture thereof.

9. The composition according to claim 1, wherein the active ingredient is the compound of formula II, pharmaceutically acceptable salt thereof, prodrug thereof, or a mixture thereof.

10. The composition according to claim 1, wherein the active ingredient is abaperidone, pharmaceutically acceptable salt thereof, prodrug thereof, or a mixture thereof.

11. A method, comprising administering the composition as claimed in claim 1 to a human.

12. A method for treating or preventing at least one selected from the group consisting of social phobia, general anxiety disorder, major depression, atypical depression, schizophrenia, psychotic disorder, and combinations thereof, comprising administering the composition as claimed in claim 1 to a subject.

13. The method according to claim 12, wherein the active ingredient is adatanserin.

14. The method according to claim 12, wherein the active ingredient is the compound of formula I.

15. The method according to claim 12, wherein the active ingredient is the compound of formula II.

16. The method according to claim 12, wherein the active ingredient is abaperidone.

17. A method of preparing the composition according to claim 1, comprising: (1) preparing a core by compressing a mixture comprising one or more of the active ingredients and a mixture of the high or medium viscosity hydroxypropylmethylcellulose (HPMC) and the high or medium viscosity hydroxyethylcellulose (HEC); and (2) optionally coating the core.
